(19) United States
(12) Patent Application Publication
(10) Pub. No.: US 2009/0291119 A1
(43) Pub. Date: Nov. 26, 2009

(54) POLYMERIC IMPLANT AND A PROCESS FOR OBTAINING A POLYMERIC IMPLANT

(75) Inventors: Jefter Fernandes Nascimento, Sao Paulo City (BR); Wagner Mauricio Pachekoski, Sao Carlos (BR)

Correspondence Address: DARBY & DARBY P.C., P.O. BOX 770, Church Street Station, New York, NY, 10008-0770, US

(73) Assignee: PHB INDUSTRIAL S.A., Serrana (BR)

(21) Appl. No.: 12/161,613

(22) PCT Filed: Feb. 6, 2007
(86) PCT No.: PCT/BR07/00028

§ 371 Date: Jan. 20, 2009

(30) Foreign Application Priority Data
Feb. 6, 2006 (BR) PI0600473-3

(51) Int. Cl. A61F 6/06; A61K 31/57; A61P 15/00

(52) U.S. Cl. 424/430; 514/178

(57) ABSTRACT

The polymeric implant is obtained from a renewable source, comprising a body formed by at least one biodegradable polymer selected from the group consisting of polyhydroxyalcanoates (PHAs) and copolymers thereof, said body incorporating, in its micro-structure, an active ingredient in a sufficient quantity to control at least one oestrus cycle of mammal animals. The invention further refers to a process for obtaining a polymeric implant comprising the steps of: mixing progesterone or progestogen, at least one polymer of the group of PHAs, one polymeric additive defined by poly(ε-caprolactone), and at least one dispersant additive and/or solvent, to obtain a colloidal solution; evaporating the solvent of the colloidal solution, and forming a paste with the components dispersed therein; drying the paste to remove the residual solvents and control the granulometry of the polymer composite; extruding the polymeric composite and moulding the latter in an implant body.

### FIELD OF THE INVENTION

The present invention refers to a polymeric implant for controlling the release of progestogens, whose composition allows said polymeric implant to present biodegradable and biocompatible characteristics, in order to optimize reproduction techniques, such as artificial insemination and embryo transfer.

The present invention further refers to a process for obtaining said polymeric implant to improve the synchronism of eostrus cycle of mammal animals.

### PRIOR ART

There are known from the prior art the devices and methods for controlling the oestrus cycle of animals, particularly bovine and ovine females of economic interest. The development of these methods for controlling animal oestrus cycle started in the decade of 1960. In this period, the intention was to establish an artificial luteinic phase by administering exogenous progestogens through several routes, such as oral, subcutaneous and vaginal topic applications.

From this period, the control of the oestrus cycle by using vaginal devices, which release progesterone (P4), received much attention of researchers around the world. Several hormonal combinations were tested and resulted in efficient protocols for synchronization of oestrus and even of ovulation, providing increments in the use of Artificial Insemination (AI) in bovine, ovine and caprine herds.

The protocols of oestrus synchronization, in which are used the vaginal devices impregnated of P4, are based on the capacity of this hormone to inhibit the oestrus and the ovulation during the period in which the devices remain in the animals. Thus, the removal of the devices causes the inhibition of the oestrus, allowing the treated animals to manifest oestrus followed by ovulation in a short period of time, in which the Artificial Insemination can be performed in a synchronized manner. The inhibition of the ovulation through P4 occurs by suppressing the release of luteinizing hormone (LH). At the initial oestrus phase of the cow (pro-estrus phase), the P4 concentrations in the blood are low and, therefore, the hypophysis releases LH in a higher frequency than during the luteinic phase, in which the P4 contents are high. During and after the luteolysis, the concentrations of P4 are reduced to undetectable levels and there is a significant increase of the LH frequency. This increase results in greater concentrations of estradiol which will induce the pre-ovulatory peak of LH and the ovulation. It should be emphasized that the estradiol only induces the peak of LH in the absence of P4 and, therefore, the animals treated with exogenous progesterone will not ovulate until the removal of the vaginal devices. Since the devices can remain for periods ranging from 7 to 10 days and are removed from all the animals at the same time, the ovulation are then released to occur in a synchronized manner.

During this period, the development and the use of materials for vaginal insertion were targets of the researchers due to the facility of application and better results of fertility.

Further during the decade of 1960, polyurethane foam impregnated with progesterone were developed, which had not proved to be viable for commercial use, since the retention rate varies greatly among the females, and there is also the possibility of infections which, in spite of not interfering with the fertility, requires the use of antibiotics.

In 1970, developed the intra vaginal pessaries were developed, known as “intra vaginal progesterone release devices (PRID)”, comprising a stainless steel helical coil surrounded by silicone impregnated with progesterone. The removal of this device from the vagina was carried out through a nylon cord fixed to the end of the helical coil.

The commercially available products consist of a silicone matrix mixed to the P4 and moulded on a support, generally made of nylon. Besides the high cost of the raw material for producing the silicone implants, the slowness in the production system of this device and the high electric energy consumption, the curing step of these products occurs at the temperature of about 200° C., raising even more the prices of the end product, and making the access of small producers to this device much more difficult.

More recently, “devices of controlled release of drugs (CIDR)” have been developed with the same silicone material, but T-shaped and with several sizes to be used in cows, goats, sheep and mares.

Nowadays, there is a system of high technology for electronic controlled release, known as “intelligent breeding device (IBD)”, developed for synchronizing the cow oestrus cycle. This system releases progesterone, estradiol and prostaglandin in predetermined periods.

All of these silicone devices, apart from the high production cost, which reflects on the end product, present a very high hormonal release index in the initial days of treatment, resulting in excess of hormone that damages the tissues and body fluids of the animal.

Up to now, the use of the known techniques, systems and methods for synchronizing oestrus cycle has been considered low, due to the reduced index of animal production and the high costs of these artificial insemination and embryo transfer techniques. The main reasons the herd breeders allege for not adopting these synchronization methods are: the need for manipulating the animal to insert and remove the pessaries, the low retention rate of the pessaries in the vagina, the possibility of infection and the lack of information about the advantages of synchronizing the oestrus and the artificial insemination in a fixed time.

There are further proposed in the prior art devices obtained from collagen matrices for controlled release of progesterone, which present a reduced production cost and a high medical and pharmaceutical application, due to their biocompatibility. However, it has been proved that the release of kinetics of these devices presents an anomalous diffusion, in which there is a relation of dependence between the concentration of the agent to be released and the time of release.

Although this system is promising, the optimization in releasing an ideal concentration of progestogens, in a predetermined period of time, requires the utilization of a higher quantity of the additive hormone in use, thereby raising the treatment process cost, and also causing hormone waste and hormone accumulation in the animal tissues.

Another disadvantage of the currently known prior art is the impossibility of releasing, in a progressive and homogeneous way, specific amounts of the hormone, in greater or lesser degree, both in the beginning and in the end of the treatment, with no risk of overloading the matrix with excessive hormone doses.

A desirable characteristic for said implants is its biodegradability. It is also desirable that the material of the implant can be biodegradable and also recyclable, so as to reduce, whenever possible, the volume of disposable material and the costs related to control procedures.

Accordingly, taking into account the inconveniences described above, the polymeric implant that controls the oestrus cycle of animals should be produced in a biocompatible and biodegradable material and present a sustained and homogeneous release kinetics in any of the treatment phases, and should not present toxicity or residues in the meat or milk of the animals, and also optimize both the reproductive techniques and the animal production.

### SUMMARY OF THE INVENTION

By reason of the inconveniences presented by the known implant devices for releasing steroids, the present invention aims at providing a polymeric implant that controls the ovulation in mammal animals, constructed by a combination of materials which are sufficiently strong, biocompatible and biodegradable and capable to contain and release, in a constant and homogeneous way, at least one effective dose of a steroid to be utilized for promoting, due to its micro-structure, the regulation of the release kinetics of the hormones to control the oestrus cycle and enhance the fertilization of the animals.

It is a complementary object of the present invention to provide a polymeric implant, as mentioned above, and which can contain, in its structure, different quantities of progesterone (P4) or progestogens to be progressively released to the animal, in the different phases of the implant action.

It is another object of the present invention to provide a process for producing the polymeric implant as cited above.

These and other objects of the present invention are achieved through the provision of a polymeric implant for controlled release of progesterone, presenting a matrix defined by a body, whose composition comprises biodegradable components presenting a proportion/contents of polymers in such a way as to enable, as a function of its micro-structure, the control of hormonal residues during the period of treatment, which implant is formed by said polymeric composition and can be reprocessed or reutilized in another animal. According to the invention, the polymeric implant is obtained from a renewable source and comprises a body formed by at least one biodegradable polymer selected from the group consisting of polyhydroxyalkanoates (PHAs) and copolymers thereof, said body incorporating, in its micro-structure, an active ingredient in a sufficient quantity to control at least one oestrus cycle of a mammal animal.

Further according to the present invention, the polymeric implant is obtained by means of a process that comprises the steps of: mixing, under controlled heating, one active ingredient selected between progesterone and progestogens; at least one polymer selected from the group of PHAs, one polymeric additive defined by poly(ε-caprolactone), and at least one dispersant addictive and/or solvent, to obtain a colloidal solution; submitting the colloidal solution to a pressing/filtrating operation, under heating, in order to promote the evaporation of the solvent and formation of a paste with the components dispersed therein; submitting the paste to a vacuum drying operation, to remove the still existing residual solvents and control the granulometry of the polymeric composite; submitting the polymeric composite to at least one extrusion operation; and moulding the polymeric composite to form an implant body according to different ways of application, through a process of injection.

The proposed process makes the matrix of the implant, in the form of a body, present effective proportions of PHB and poly(ε-caprolactone)(PCL), so as to control the hormone dispersion as a function of the period of time and of the temperature in the different steps of implant usage. Said implant also presents a more homogeneous release profile that avoids excessive quantities of the hormone to be released in the first days of use, without jeopardizing the release in the final days of the treatment, besides the release kinetics that can be controlled by several other mechanisms, such as the micro-structure of the polymers that form the implant, the porosity of said implant resulting from its micro-structure, the alteration of the polymer proportion, and also the inclusion of additives with different functions.

### BRIEF DESCRIPTION OF THE DRAWINGS

The invention will be described below, with reference to the enclosed drawings, given by way of example of one embodiment for the polymeric composite and in which:

FIG. 1 illustrates a rear lateral perspective view of a possible embodiment for the polymeric implant of the present invention;

FIG. 2 represents a comparative graph of the profile of in vitro release of progesterone from the implant of the present invention in relation to a prior art product;

FIG. 3 represents a graph of the daily in vitro release of progesterone (P4), from the implant of the present solution.

FIG. 4 represents a graph of the daily release of in vivo progesterone (P4), from the implant of the present solution.

FIG. 5 represents a comparative graph of the average of the in vitro release of progesterone from the implant of the present invention in relation to a prior art product (DID) (implant of Argentinean silicone); and

FIG. 6 represents a graph illustrating the concentration levels of progesterone in the animal blood of the polymeric implant of the present invention in relation to a prior art product (DID).

### DETAILED DESCRIPTION OF THE INVENTION

In one aspect of the present invention there is provided an intra vaginal implant, of a variable geometric shape, to be applied in the interior of the vaginal cavity of an animal, and retained in the cavity over the period of time within the range from 7 to 12 days and then removed from said cavity to permit the occurrence of the oestrus and ovulation, said implant comprising a body formed by a biocompatible and biodegradable polymeric composition, dimensioned so as to incorporate and disperse a determined concentration of hormone or progestogen, and which also retains the hormonal additive when desired. The implant in polymeric material can be produced through several processes, such as for example moulding, generally by injection, at least one biodegradable polymer, which can be selected from the group consisting of polyhydroxyalkanoates (PHAs), polyhydroxybutyrate (PHB) and polyhydroxyvalerate (PHBV), said polymeric implant being constructed to present the density and the structure of its walls dimensioned to lead to an interfacial adhesion as a function of the secondary intermolecular interactions of both the biodegradable polymers and the hormone.

The process of preparing the implant of the present invention utilizes, as a structural matrix, biodegradable polymers obtained from polyhydroxyalkanoates (PHAs), between which can be selected from poly-3-hydroxybutyrate (PHB), poly (hydroxybutirate-co-hydroxyvalerate) (PHBV) or mixtures of these polymers and copolymers.

Polyhydroxyalkanoates (PHAs) are biodegradable thermoplastics and, furthermore, they are biocompatible and have been recognized as potential substitutes for petroleum-derived thermoplastics.

The degradation rates of these articles, under several environmental conditions, are of great relevance for the user of articles made of PHB or copolymers thereof. Poly (3-hydroxybutyric-co-hydroxyvaleric acid)—PHBV. Biodegradation usually occurs via surface attack by bacteria, fungi and algae. The actual degradation time of biodegradable polymers and, therefore, of the PHB and PHBV, will depend upon the surrounding environment, as well as upon the thickness of the articles.

The reason that makes these polymers acceptable as potential biodegradable substitutes for the synthetic polymers is their complete biodegradability in aerobic and anaerobic environments to produce CO₂/H₂O/biomass and CO₂/H₂O/CH₄/biomass, respectively, through natural biological mineralization.

One of the members of this class, the Poly (3-hydroxybutyric acid) or PHB, was mentioned in the microbiology literature in the beginning of the 20^{th} century. Detailed studies were reported by Maurice Lemoigne, of Pasteur Institute, in 1925. He noticed insoluble grains included in the cytoplasmic fluid of Bacillus megaterium culture medium, which are common in the case of lipids, and after several characterizations, it has proved to be a polyester having an empiric formula (C₄H₆O₂).

Inside the cell, the polymer acts as a source of energy and carbon, remaining in the amorphous or not crystalline state. However, in the process of extraction, the crystallization occurs rapidly, leading to high crystallinity levels and therefore of rigidity, which has made highly difficult the use of this polymer due to its low impact strength.

Due to its natural origin, the PHB has an exceptional stereochemical regularity; its chains are linear with interactions of the van der Waals type between carbonyl oxygen and the methyl groups and interaction through hydrogen bridges due to the presence of hydroxyls. The chiral centers have only the configuration R, which means the polymer is completely isostactic and thus highly favorable to crystallization.

The structural formulas of the -3-hydroxybutyric acid monomer and of PHB polymer are illustrated below:

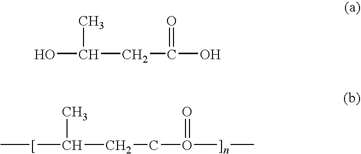

In order to achieve a specific processing-structure-properties-cost relationship, the polymeric composition, besides the PHB and/or its copolymers, can contain variable contents of the biodegradable polymer poly(caprolactone)—PCL and additives.

In order to reduce the degradation caused by the severity of the aggressive agents (shearing, temperature and oxygen), in the processing of the polymeric implant, it is desirable the addition of complete systems of thermal stabilization consisting of: primary antioxidant of the sterically hindered phenol type (in contents from 0.02% to 0.5%-% in mass regarding the total content, which includes the PHB and the PCL); secondary antioxidant of the organic phosphite type (in contents from 0.02% to 0.5%-% in mass regarding the total content, which includes the PHB and the PCL); thermal stabilizers of the lactone type (in contents from 0.02% to 0.5%-% in mass regarding the total content, which includes the PHB and the PCL).

In order to obtain a complementary interaction, it is possible to utilize secondary co-stabilizers of the process auxiliary type (internal lubricant, external lubricant and flow modifiers).

For the thermodynamic and kinetic control of the crystallization process (nucleation and growth) of the PHB and of the PCL, in the polymeric compositions, it is possible to utilize the nucleants sorbitol or sodium benzoate. According to the desired crystalline morphology and crystallinity degree, the nucleant content must be varied, in a combined form with the cooling gradient imposed to the polymeric material during its processing final stage.

### Hormone for Pharmaceutical Control of the Animal Reproduction—Progesterone:

Progesterone (P4) is a steroid hormone and is the main component in the regulation of the female reproductive function. In general, the main effects of the P4 in the mammals are:

1) in the uterus and ovary: participation in the mechanism which results in the release of mature oöcytes, facilitation of the conceptus implantation and pregnancy retention, by promoting the uterine growth and suppression of the myometrium contractility.
2) in the mammary gland: lobule-alveolar development in the preparation for milk secretion and suppression of protein synthesis of the milk before delivery.

3) in the brain: measurement of signals required for manifestation of the sexual behavior.

The ovary is the main place where the progesterone and the estradiol are synthesized in mammals.

These hormones are synthesized in such a way as to provide cyclic fluctuations of their contents in the blood stream. Before the ovulation, the granulose cells of the follicles synthesize and secret estrogen. After the ovulation, these granulose cells undergo a maturation process and form the luteous body (LB) which is responsible for the secretion of P4 in the subsequent phase of the cycle.

If there is no fertilization, the luteous body continues to grow over 10-12 days and suffers regression, therefore ceasing to secrete P4. In case fertilization occurs, the LB continues to grow and maintains its function for 2 or 3 months of gestation. After this period, it recedes gradually and the placenta assumes the role of synthesizing hormones (P4 being one of them) to maintain the pregnancy.

Alternatively, the progesterone (P4) can be substituted by progestogens. Progestogens are synthetic hormones whose action is very similar to the P4 action. In some situations, the employment of progestogens can be advantageous, since they are much more potent than P4, requiring the administration of significantly lower doses. This enables to construct smaller devices that can be administered not only in the vaginal cavity, but also implanted subcutaneously, in any part of the animal body, but preferably in the auricular pavilion, in the case of animals whose meat is destined for human consumption. Progestogens, such as P4, also inhibit the ovulation and are the elective drugs when contraception is desired. Among progestogens, it is possible to employ medroxyprogesterone, melengestrol acetate, megestrol acetate, norgestomet, levonorgestrel, gestodene, fluorogestone acetate and others.

### Methodology of Production of the Polymeric Composite

For the incorporation and complete dispersion of the additive hormone in the biodegradable polymeric implant, an economically viable system has been developed for preparing the polymeric composition, which enables the manufacture of moulded articles (extrusion and injection).

The additive hormone, in its natural physical form, cannot be incorporated directly into polymeric matrices, due to the alterations suffered by the additive in its physical-chemical properties. Thus, it is fundamental to convert the system into a colloidal solution. The solvent evaporation allows forming a system with an additive that is totally dispersed and surrounded by the polymeric matrix through an interfacial adhesion mechanism, due to the secondary intermolecular interactions, biodegradable polymers—hormone.

This methodology permits the use of biodegradable polymers from renewable sources, mainly in applications where thermal resistance is required, either during manufacture of the moulded component or during the step of applying this component.

In the method of the present invention, the technological parameters, during the step of forming the colloidal solution, include adding and mixing, in a mixing equipment with blades or helices, the biodegradable polymers, additives and a solvent. Thus, a mixture of the components in a colloidal solution will be produced.

During the pressing/filtrating step, the technological parameters include, in particular, the pressing temperature, for example, temperatures from 70° C. to 90° C., and the conditions of pressure and time during the process. It is important to watch the phases of heading, discharge, heating curve or profile, and assembly calibration.

The technological parameters are optimized as a function of the basic formulation of the polymeric composite, as well as of the characteristic properties of the raw materials. The quantity of solvent for hormone additive dissolution is of major importance for the composite properties, with the addition of dispersant additives collaborating for an improved homogenization of the system, with consequent improvements in the final properties of the product.

The mixture of the raw materials is carried out in a mixer provided with blades or helices, with high or medium rotation speed, and preferably with controlled heating system. All the raw materials, such as: biodegradable polymers (PHB, and/or its copolymers, and PCL), hormone additive, solvent, dispersant additives and other additives are inserted in the mixer, as exemplified below.

After the formation of the colloidal solution, the pressing/filtrating step is carried out in a system heated for total evaporation of the solvent with consequent formation of the “paste”. The resulting material of this process is sent to a vacuum drying system for removal of the residual solvents which can be dispersed in the polymeric implant. During the pressing step, it is possible, through technological parameters, to control the granulometry of the polymeric composition, determining and/or altering its final thermo-mechanical properties.

The process for preparing the polymeric composites can be optionally substituted for a physical pre-mixture of the components of the developed formulation, in the solid state and with a suitable temperature control.

In the sequence of the process, the granules of the polymeric composite are manufactured by extrusion.

It is recommended the employment of a Twin-Screw Extruder Co-Rotating Intermeshing containing Gravimetric Feeders/Dosage Devices of high precision.

In one example of carrying out the invention, extrusion was responsible for producing the composites and their granulation. A modular screw profile with transport elements (left/right handed) was used to control the pressure field, and kneading elements (kneading blocks) to control the fusion and the mixture. This group of elements has proved to be a primordial factor for achieving a suitable morphological control of the structure and a good dispersion of the hormone and of the additives in the polymeric composition.

Table 1 below presents the extrusion conditions for the PHB/PCL/Hormone/Additives polymeric compositions.

TABLE 1

| | Temperature (° C.) | | | | | | | Speed |
|---|---|---|---|---|---|---|---|---|
| Composite | C1 | C2 | C3 | C4 | C5 | Matrix | (melt) | (rpm) |
| PHB/PCL/ | 125-135 | 130-135 | 150-155 | 135-145 | 145-155 | 150-155 | 150-155 | 140-145 |
| Hormone/ | | | | | | | | |
| Additives | | | | | | | | |

The modification and the incorporation of the hormone to the polymeric composite was continuously carried out in one stage comprising the following steps:

a) continuously adding polymeric composite and, if desired, solid or liquid additives in the first extrusion zone for feeding and start mixing.
b) heating and compressing the polymeric composite in the second extrusion zone.
c) heating and mixing in the third extrusion zone for forming the melt.
d) compressing and mixing in the fourth and fifth extrusion zones, for homogenization of the melt, by applying a vacuum degassing system to eliminate residual humidity of the polymeric composition.
e) extruding the melt through an extrusion matrix with subsequent cooling in water at ambient temperature granulation, and packaging.

### Moulding by Injection:

In order to manufacture the body 10 of the device for controlling the animal reproduction, in its different forms, it is necessary to utilize the process of injection, in which the polymeric composite is moulded according to the geometry required for the different applications.

It is necessary to utilize an injecting machine which enables an adequate control of the temperature and which is apt to receive the mould for production of the devices.

Table 2 below presents the injection conditions of the PHB/PCL/Hormone/Additives polymeric compositions.

TABLE 2

| Temperature Profile (° C.) | Pressure Profile/Times |
|---|---|
| Zone 1: 150-155 | Pressure (bar): 350-400 |
| Zone 2: 155-160 | Pressurization (bar): 360-390 |
| Zone 3: 170-175 | Flowrate (cm³/s): 15-25 |
| Zone 4: 170-175 | Holding (bar): 280-320 |
| Zone 5: 170-175 | Holding time (s): 10-15 |
| Mould (° C.): 30-40 | Counter pressure (bar): 35-45 |
| Cooling time (s): 30-35 | Dosing speed (mm/min): 10-15 |

### Formulation of the Polymeric Composition

The PHB can be a homopolymer and/or its copolymers with valerate—P(HB-HV), with contents of valerate between 5% and 40%, having molecular weight between 10,000 and 1,200,00 Da, but preferably, between 200,000 and 600,000 Da.

The PHB and/or its copolymers can further be added with of variable quantities of PCL, between 5 and 60%, but preferably between 40 and 50%. PCL must have a molecular weight between 10,000 and 800,000 Da, but preferably, between 100,000 and 500,000 Da.

### Progesterone (P4) as Active Principle:

The contents of P4 can range from 5 to 200%, preferably from 8 to 10%.

Other progestogens, such as medroxyprogesterone acetate, fluorogestone acetate, melengestrol acetate, levonorgestrel, norgestomet or gestodene, can be alternatively employed, in concentrations that can range from 1 to 20%.

### Additives for Thermal Stability:

In order to reduce the degradation caused by the severity of the aggressive agent (shearing, temperature and oxygen), in the processing of the polymeric composites, one should promote the addition of complete systems of thermal stabilization consisting of: primary antioxidant of the sterically hindered phenol type (in contents from 0.02% to 0.5%-% in mass regarding the total content, which includes the PHB and the PCL); secondary antioxidant of the organic phosphite type (in contents from 0.02% to 0.5%-% in mass regarding the total content, which includes the PHB and the PCL); thermal stabilizers of the lactone type (in contents from 0.02% to 0.5%-% in mass regarding the total content, which includes the PHB and the PCL).

In a complementary interaction, one can utilize secondary co-stabilizers of the process auxiliary type (internal lubricant, external lubricant and flow modifiers), in contents from 0.5% to 3%.

For the thermodynamic and kinetic control of the crystallization process (nucleation and growth) of the PHB and of the PCL, in the polymeric compositions, the nucleants sorbitol or sodium benzoate can be used. According to the desired crystalline morphology and crystallinity degree, the nucleant content (between 0 and 0.25%) should be varied in a combined form with the gradient of cooling imposed to the polymeric material during its processing final stage.

### Kinetics of In Vitro Release and Geometry of the Device

In terms of product innovation, the polymeric composites described herein enable a very homogeneous release of hormones, with the advantage of avoiding an excessive release in the beginning of the treatment, which means hormone waste with evident cost increase. Moreover, it has recently been verified that zebu calves had its follicular development altered, due to the high plasmatic contents of progesterone provided by one of the commercially available products for sustained release of the hormone. In this work, zebu calves (Bos taurus indicus) presented lower growth rate and lower maximum diameter of the dominant follicle, lower ovulation rate and greater plasmatic concentration of progesterone than calves of European origin (Bos taurus taurus), when treated with the vaginal device CIDR. The conclusion was that the progesterone contents provided by the commercially available vaginal devices, although adequate for animals of European origin, can be excessive and even harmful to the fertility of zebu bovine females. Indeed, the commercially available products had as a goal the retention of the plasmatic contents of progesterone in the order of 2 ng/mL and, when utilized in zebu calves, provide contents of about 5.4 ng/mL, impairing the fertility of these animals. It has been established that a product, to be more adequate for use in zebu cows, must release less quantity of P4, in order to provide plasmatic contents closer to the ideal.

The release of the progesterone from the devices of the present invention predominantly occurs by the diffusion mechanism, since the time necessary for the biodegradation to significantly contribute to release the hormone is much greater than the time of permanence of the device in the animal, in order to obtain the desired therapeutic effects.

Accordingly, the intermolecular space in the micro-structure of the polymeric composite is an important mechanism for regulating the release kinetics of the hormones. The diffusion of the progesterone in the intermolecular space of the micro-structure of these composites can be controlled by the inclusion of polycaprolactone, in proportions that can range from 5 to 60%. In this case, the restriction of mobility of the progesterone in the resulting blends is lower and, consequently, the diffusion of the progesterone is greater in the resulting blends, as compared to both polymers separately.

Other mechanism through which the release speed can be controlled concerns the molecular weight (PM) of the PHB employed. The biodegradable polymers PHB and the copolymers P(HB-HV) of molecular weights between 10,000 and 1,200,000 Da have proved to be useful in the manufacture of the vaginal auricular and subcutaneous devices. The lower the PM of the PHB utilized in the composite, the higher the release speed of P4 and progestogens.

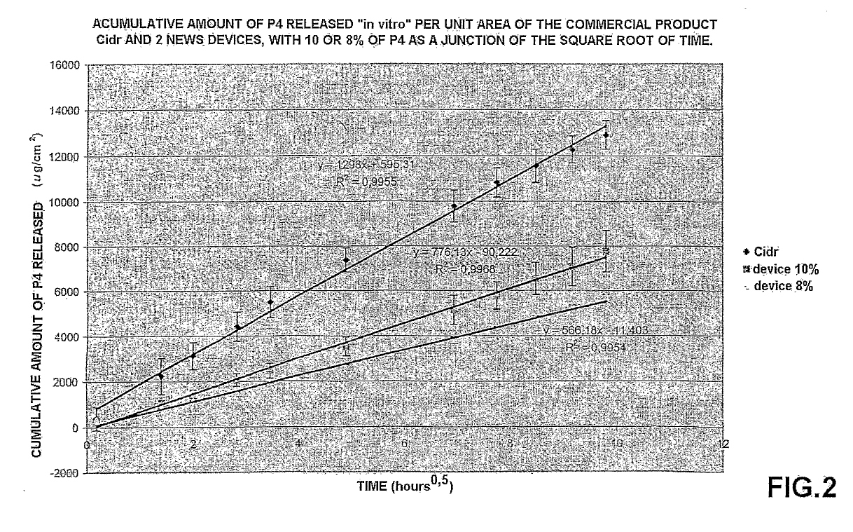

The profiles of in vitro release of progesterone through the polymeric composite of the present solution in relation to one of the commercial products can be seen in FIG. 2.

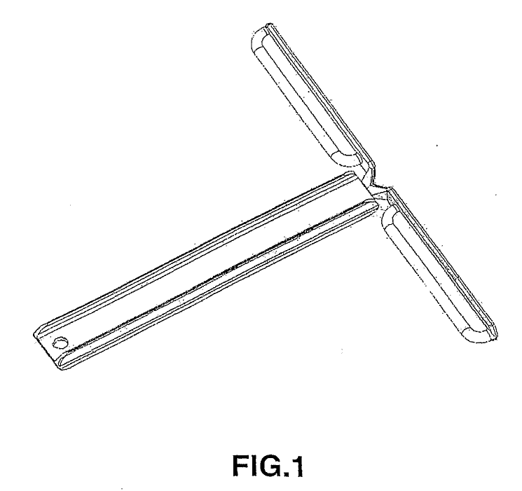

Preferably, the vaginal devices can be moulded in the format showed in FIG. 1. The contact area with a vaginal mucosa can range from 70 to 200 cm², but should be preferably situated between 120 and 150 cm².

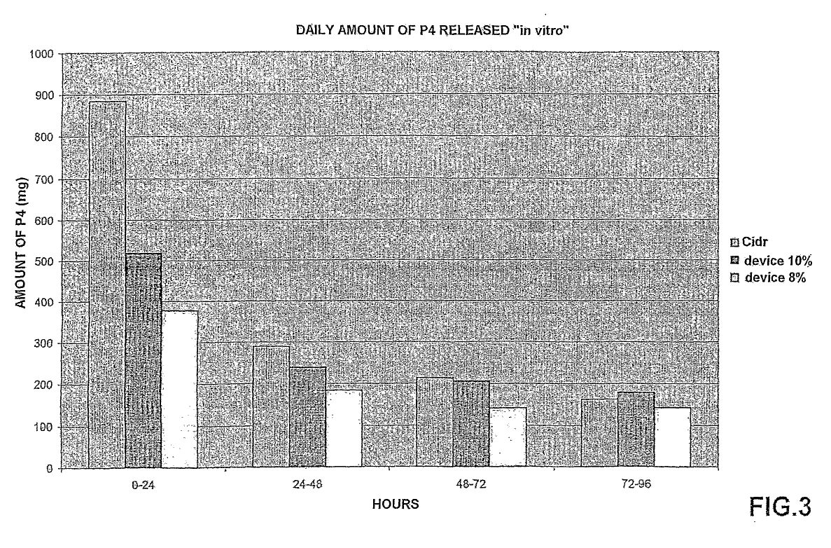

The quantities of P4 released every 24 hours, from 2 minutes to 96 hours of in vitro experiment, can be seen in FIG. 3.

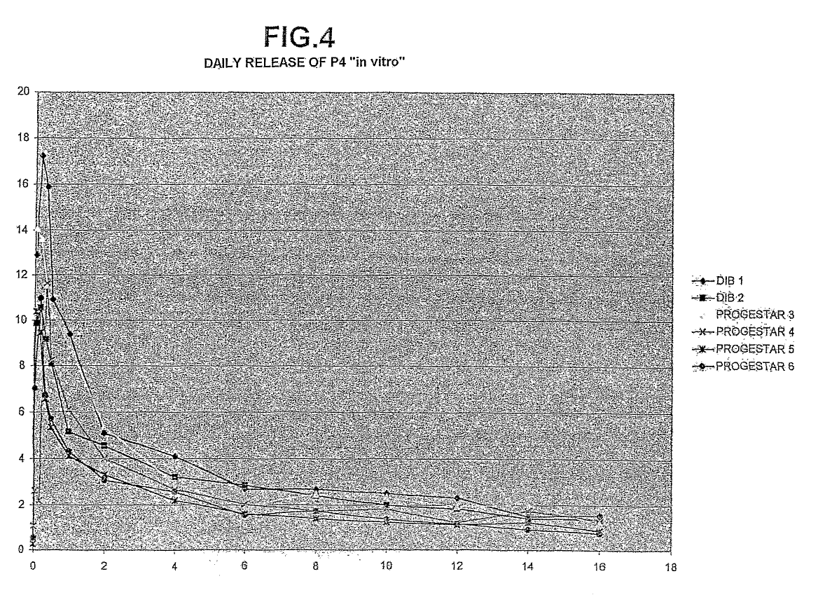

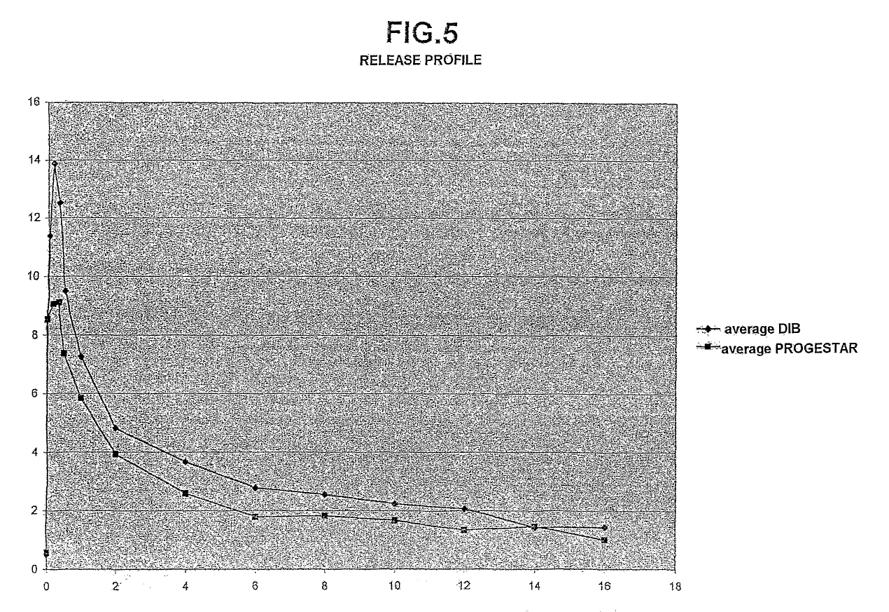

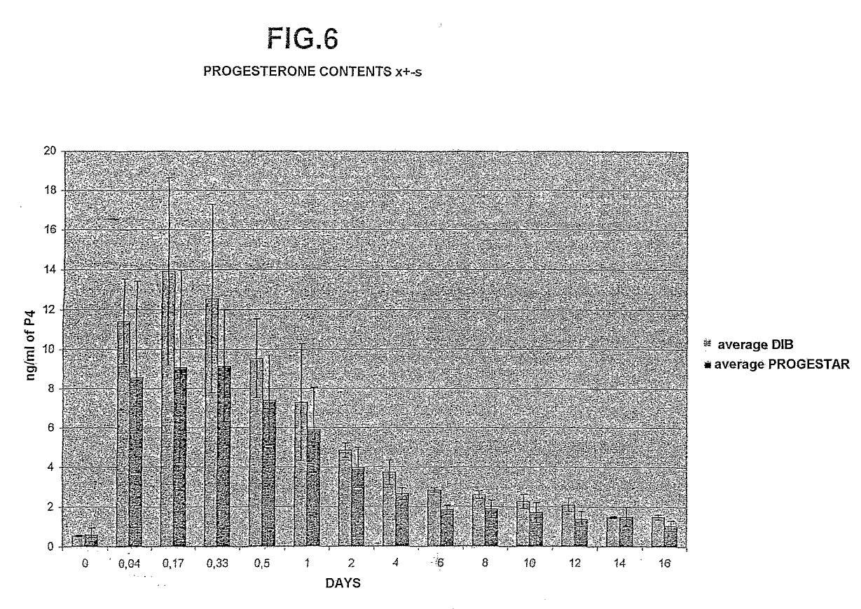

FIGS. 4, 5 and 6, as already mentioned above, represents graphs that illustrate the characteristics of in vitro release of progesterone from the implant of the present invention in relation to a silicone implant constructed according to the prior art.

The device can have or not a support frame for the layer of the polymeric composite incorporated to the hormone, but preferably, the support frame is not employed. The auricular implants are preferably cylindrical, with diameter from 2 to 3 mm, and variable length, preferably between 1 and 4 cm.

The product of the present invention can be produced by the process of extrusion or injection, with greater yield and lower electric energy consumption.

As a function of the aspects emphasized above, the present invention has advantages in relation to the prior art, since the polymeric implant employed is produced in biocompatible and biodegradable material, while those of the prior art, produced in silicone, are only biocompatible. Furthermore, the implant of the present solution has more suitable mechanical properties for the desired use, once it can be moulded in different shapes and allows effecting the necessary deformations to facilitate the insertion, retention in the vaginal cavity and removal of the devices at the end of the treatment.

1. Polymeric implant obtained from a renewable source, wherein it comprises a body formed by at least one biodegradable polymer selected from the group consisting of poly-3-hydroxybutirate (PHB), poly-(hydroxyburate-co-hydroxyvalerate) (PHBV), and mixtures of these polymers and copolymers, said body comprising, in its micro-structure;
an active ingredient in a sufficient quantity to control at least one oestrus cycle of mammal animals;
at least one primary antioxidant of the sterically hindered phenol type;
at least one secondary antioxidant of the organic phosphate type;
one additive of thermodynamic and kinetic control of crystallization; and
at least one thermal stabilizer.

2. Polymeric implant, as set forth in claim 1, wherein the implant body further comprises a polymeric additive defined by poly(ε-caprolactone) (PCL).

3. Polymeric implant, as set forth in claim 2, wherein the implant body comprises from about 5% to 60% of poly (ε-caprolactone) (PCL).

4. Polymeric implant, as set forth in claim 3, wherein the implant body presents a molecular weight from about 10,000 to 800,000 Da.

5. Polymeric implant, as set forth in claim 1, wherein the implant body comprises PHB and about 5% to 40% of valerate.

6. Polymeric implant, as set forth in claim 5, wherein the implant body presents a molecular weight from about 10,000 to 1,200,000 Da.

7. Polymeric implant, as set forth in claim 1, wherein the implant body comprises from 0.02% to 0.5% in mass of the primary antioxidant and from 0.02% to 0.5% in mass of the secondary antioxidant.

8. Polymeric implant, as set forth in claim 1, wherein the implant body comprises from 0.02% to 0.5% of at least one thermal stabilizer of the lactone type.

9. Polymeric implant, as set forth in claim 1, wherein the implant body further comprises from 0.05% to 3% of at least one secondary co-stabilizer of the process auxiliary type, constitutive of any one of the additives—internal lubricant, external lubricant and flow modifiers.

10. Polymeric implant, as set forth in claim 1, wherein it comprises from about 0% to 0.25% of the additive of thermodynamic and kinetic control of crystallization selected from nucleants of sorbitol or sodium benzoate types.

11. Polymeric implant, as set forth in claim 10, wherein the implant body further comprises a polymeric additive defined by poly(ε-caprolactone) (PCL).

12. Polymeric implant, as set forth in claim 11, wherein the implant body further comprises from about 5% to 60% of a polymeric additive defined by poly(ε-caprolactone) (PCL).

13. Polymeric implant, as set forth in claim 12, wherein the implant body presents a molecular weight from about 10,000 to 800,000 Da.

14. Polymeric implant, as set forth in claim 1, wherein the active ingredient is selected from progesterone and progestogens, in a concentration ranging from about 5% to 20% by weight of the implant body.

15. Polymeric implant, as set forth in claim 14, wherein the progestogens are selected from the group consisting of medroxyprogesterone acetate, fluorogestone acetate, melengestrol acetate, 30 levonorgestrel, norgestomet and gestodene, employed in a concentration ranging from about 1 to 20% by weight of the implant body.

16. Process for obtaining a polymeric implant, wherein it comprises the steps of:
mixing, under controlled heating, one active ingredient selected from progesterone and progestogens; at least one polymer selected from the group of PHAs, one polymeric additive defined by poly(ε-caprolactone), and at least one dispersant additive e/or solvent, to obtain a colloidal solution;
submitting the colloidal solution to a pressing/filtrating operation, under heating, so as to promote evaporation of the solvent and formation of a paste with the components dispersed therein;
submitting the paste to a vacuum drying operation to remove the still existing residual solvents and control the granulometry of the polymeric composition;
submitting the polymeric composite to at least one extrusion operation; and
moulding the polymeric composite to form an implant body according to different ways of application, through a process of injection.

17. Process, as set forth in claim 16, wherein the pressing/filtrating operation of the colloidal solution is carried out under temperatures of about 70″ to 90° C.

18. Process, as set forth in claim 16, wherein the polymer is selected from the group consisting of poly-3-hydroxybutirate (PHB), poly-25 (hydroxyburate-co-hydroxyvalerate) (PHBV), mixtures of these polymers and copolymers.

19. Process, as set forth in claim 18, wherein the polymer is poly-3-30 hydroxybutirate (PHB).

20. Process, as set forth in claim 18, wherein it comprises PHB and about 5% to 40% of valerate.

21. Process, as set forth in claim 18 wherein the implant body further comprises from about 5% to 60% of the poly(ε-caprolactone) (PCL).

22. Process, as set forth in claim 16, wherein the active ingredient is present in a concentration ranging from about 5% to 20% by weight of the implant body.

23. Process, as set forth in claim 16, wherein the progestogens are selected from the group consisting of medroxyprogesterone, fluorogestone acetate, melengestrol acetate, levonorgestrel, norgestomet and gestodene, employed in a concentration ranging from about 1 to 20% by weight of the implant body.

24. Polymeric implant, as set forth in claim 2, wherein the implant body comprises from about 40% to 50%, of poly (ε-caprolactone) (PCL).

25. Polymeric implant, as set forth in claim 3, wherein the implant body presents a molecular weight from about 100,000 to 500,000.

26. Polymeric implant, as set forth in claim 11, wherein the implant body further comprises from about 40% to 50%, of a polymeric additive defined by poly(ε-caprolactone) (PCL).

27. Polymeric implant, as set forth in claim 12, wherein the implant body presents a molecular weight from about 100,000 to 500,000.

28. Polymeric implant, as set forth in claim 1, wherein the active ingredient is selected from progesterone and progestogens, in a concentration ranging from about 8% to 10% by weight of the implant body.

29. Process, as set forth in claim 18 wherein the implant body further comprises from about 40% to 50% of the poly(ε-caprolactone) (PCL).

30. Process, as set forth in claim 16, wherein the active ingredient is present in a concentration ranging from about 8% to 10% by weight of the implant body.
